(19) United States
(12) Patent Application Publication
(10) Pub. No.: US 2005/0112022 A1
(43) Pub. Date: May 26, 2005

(54) Room sanitizing method and apparatus

(76) Inventor: David M. Morgan, Dayton, OH (US)

Correspondence Address: DAVID A. GREENLEE, P.O. BOX 340557, COLUMBUS, OH, 43234-0557, US

(21) Appl. No.: 10/975,801

(22) Filed: Oct. 28, 2004

Related U.S. Application Data
(60) Provisional application No. 60/525,493, filed on Nov. 26, 2003.

(51) Int. Cl. A61L 2/22

(52) U.S. Cl. 422/28; 422/292; 514/642

(57) ABSTRACT

An aerosol canister containing sanitizing liquid and propellant is provided to sanitize exposed surfaces in a room in a short period of time. The canister is placed in a closed room and its dispensing valve is actuated to dispense the sanitizing liquid in an aerosol mist of droplets which expand to fill the room in mere minutes. The canister can include a weighted rounded base to enable the canister to be tossed into the room and assume an upright dispensing position. The base can be filled with weighting material just before use. One embodiment can be rolled into the room.

### CROSS-REFERENCE TO RELATED APPLICATION

Applicant claims priority of Provisional Patent Application Ser. No. 60/525493, filed Nov. 26, 2003.

### TECHNICAL FIELD

This invention relates to sanitizing surfaces subject to human contact and, more particularly, to a method and apparatus for sanitizing exposed surfaces in a room.

### BACKGROUND OF THE INVENTION

Many soaps and liquids have been developed to sanitize and, thus disinfect, surfaces subject to human contact to prevent contact with germs and the spread of disease. An example of a liquid developed for manual application to exposed surfaces, such as toilet seats, is found in U.S. Pat. No. 6,146,587, issued to the inventor herein. The liquid is sprayed onto the desired surface, where it dries on contact, disinfecting and sanitizing the surface for human contact without the risk of encountering germs.

Other surfaces which humans regularly contact are door knobs and handles, stair railings, desks, tables, computer keyboards, telephones and chair arms. All of these surfaces can be sprayed to disinfect and sanitize, although the sprayer must remember to spray all necessary surfaces. Also, this takes some time to spray all these surfaces in a house or office. However, it would be desirable to develop a method and apparatus whereby all necessary surfaces, subject to human contact, could be easily and quickly sanitized.

### SUMMARY OF THE INVENTION

It is an object of this invention to develop a method and apparatus whereby all necessary surfaces, subject to human contact, could be easily and quickly sanitized, or disinfected.

In one aspect this invention features a method and apparatus for sanitizing exposed surfaces, that are subject to human contact, in a room in a short period of time by placing an aerosol canister containing sanitizing liquid and propellant in a closed room and actuating the dispensing valve to dispense the sanitizing liquid in an aerosol mist of droplets which expand to fill the room in mere minutes.

In another aspect, this invention features an aerosol canister which contains a sanitizing composition of a didecyl dimethyl ammonium chloride and has a dispensing valve that is manually operable to open and remain open until the canister contents are dispensed in an aerosol mist of droplets throughout a closed room to coat exposed surfaces with the composition, which dries upon contact. Upon opening the room, the canister is removed, leaving behind a sanitized room.

In some instances, a contaminated room poses a health risk to anyone entering the room. It would be desirable to be able to sanitize the room without requiring the person performing the sanitizing operation to enter the unsanitized room, thus preventing the risk of contamination.

Thus, in a further aspect, this invention features such an aerosol canister which can be activated and then placed in a room without actually entering the room. Preferably, the canister can be propelled or thrown into the room from a remote location, and is constructed to always dispense the sanitizing spray upwardly.

This sanitizing process can be repeated periodically as necessary. Offices and rooms experiencing a greater frequency and number of occupants would require more frequent sanitizing than homes.

These and other objects and features of this invention will become more readily apparent upon reference to the following detailed description of a preferred embodiment, as illustrated in the accompanying drawings, in which:

### BRIEF DESCRIPTION OF THE DRAWINGS

FIG. 1 is a perspective sketch of a room being sanitized according to this invention;

FIG. 2 is a simplified side view of the room of FIG. 1, illustrating the dispersion of the contents of the canister;

FIG. 3 is a perspective view of one embodiment of a dispensing canister according to this invention;

FIG. 4 is a top view of the canister;

FIG. 5 is a partial section of the top of the canister, taken along lines 5 5 of FIG. 4, showing details of the dispensing valve;

FIG. 6 is a view similar to FIG. 4, showing the valve actuated;

FIG. 7 is a top view of another embodiment of dispenser;

FIG. 8 is a partial sectional view of the embodiment shown in FIG. 7;

FIG. 9 is a top view of yet another embodiment of dispenser;

FIG. 10 is a partial sectional view of the embodiment of FIG. 9;

FIG. 11 is a side view of still another embodiment of canister, with the cap shown in section;

FIG. 12 is an end view of the canister of FIG. 11;

FIG. 13 is an enlarged partial sectional view taken along line 13-13 of FIG. 12, with the dispensing valve shown actuated; and

FIG. 14 is a detail view of the dispensing valve.

### DESCRIPTION OF THE PREFERRED EMBODIMENT

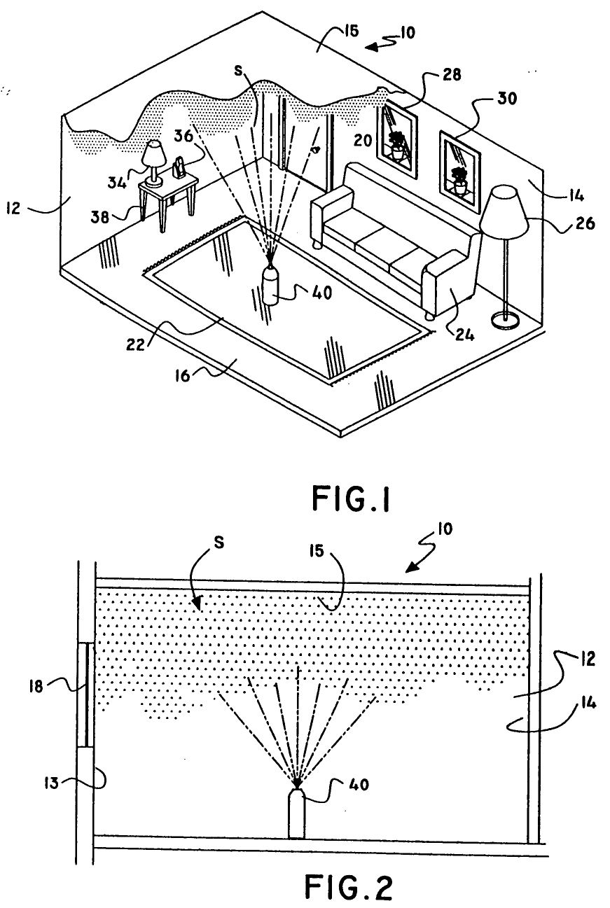

Referring to FIGS. 1 and 2, a room 10 has walls 12, 13 and 14 of painted wallboard or covered with wallpaper (other walls not shown), a ceiling 15 of painted wallboard (only partially shown), and a wood floor 16. Wall 13 includes a window 18 and wall 12 includes a door 20. A rug or carpet 22 partially covers floor 16. Room furniture adjacent wall 14 includes a couch 24 and floor lamp 26 and pictures 28 and 30. Adjacent wall 12 are a lamp 34 and telephone 36 on a table 38.

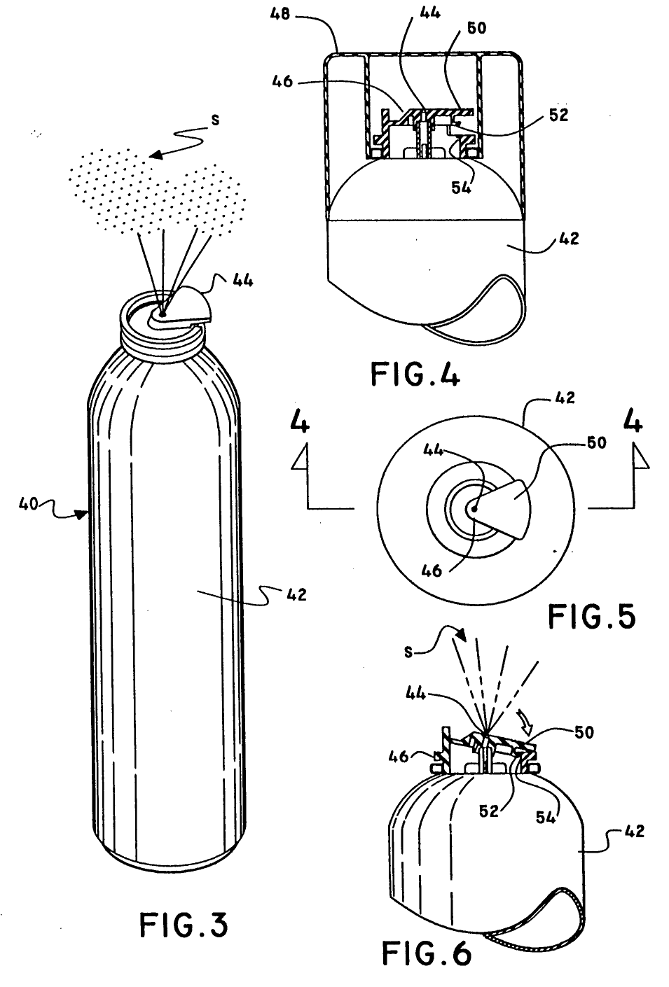

A canister 40 containing the sanitizing liquid is placed in the center of room 10 by a person who has closed window 18. As shown in FIGS. 3-6, this canister has a body 42 that mounts a dispensing valve having a spray nozzle 44 controlled by a control lever switch 46. After removing the top 48, switch 46 is actuated by depressing a flap 50 so that a tab 52 deforms and detents under a shoulder 54, which locks the nozzle open to enable the sanitizing liquid to continue to emit as an aerosol spray S until canister 10 is empty.

The person can then exit the room and close door 20 to confine the dispersing spray 28 to the closed room, as illustrated in FIGS. 1 and 2. As shown, the spray is propelled outwardly and upwardly to a height near or at the ceiling 15. The spray droplets then fall downward and impinge on succeeding upwardly propelled droplets. The net effect is a mushrooming of the spray droplets throughout the entire room, descending to contact all exposed surfaces.

Canister 10 preferably contains 8 ounces of the liquid, which is preferably a n-decyl dimethyl ammonium chloride in an alcohol diluent. It can contain an additional fragrance. The propellant is preferably a compressed gas such as pentane, propane or butane. Details of packaging and formulation can be found in U.S. Pat. No. 6,146,587, the entirety of which is incorporated herein by reference. The expelled liquid droplets are generally benign to all surfaces it contacts, whether made of fabric, wood, paint, paper, etc. and will not stain. It will, however, disinfect and sanitize these surfaces. The entire dispersal is accomplished in 2-2½ minutes, with the droplets drying upon contact. Thereafter, the room may be entered to collect and dispose of the empty canister 40. The room may now be used normally, with the assurance that exposed surfaces are sanitized.

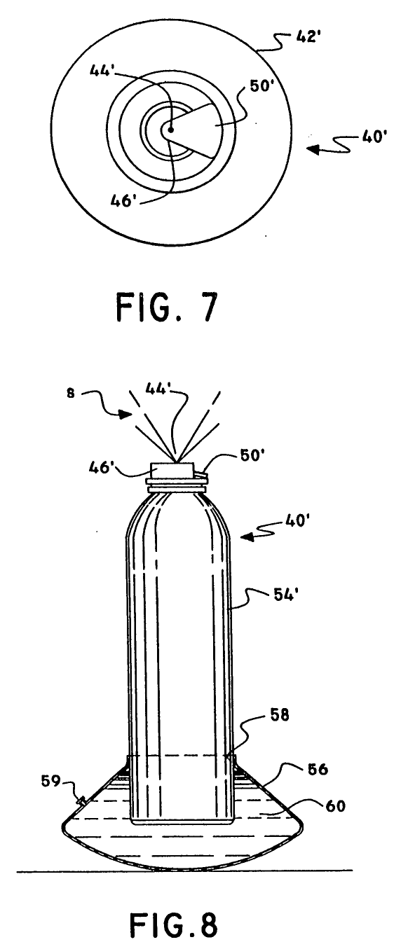

Another embodiment of dispenser is shown in FIGS. 7 and 8. Here a canister 40′ is similar to dispenser 54 previously described, with similar parts indicated by the same numeral primed. In this embodiment an enlarged weighted base 56 has a cylindrical receptacle 58 for receiving and securing the bottom of canister body 54′. Base 56 is frusto-conical in shape, with a rounded bottom that defines a cavity containing water, or other liquid, or any other heavy substance, such as sand, shot, etc. The purpose of base 56 is to provide a bottom-weighted canister that will attain an upright position, regardless of its original orientation. The base 56 can be made with a conventional closable opening 59, which enables filling with the weighting substance at a time later than manufacture. The advantage is to reduce the weight for transport, shipping and carrying, but enabling filling with the weighting substance when ready for use. This would facilitate the carrying and use of many such canisters by a person for sanitizing many contaminated rooms.

This construction of canister 40′ is advantageous in situations where a person does not want to enter the room or confined space to be sanitized. In this situation, the room door can be opened and the valve 44′ of weighted canister 40′ actuated and the canister is placed by manually or mechanically propelling or tossing it into the room. With the weighted base 56, gravity will cause canister 40′ to attain the upright position of FIG. 8, regardless of its original orientation after being tossed. Although initially rocking, it will settle, assuring that the contents are discharged upwardly as spray S, as shown in FIG. 1, for even dispersion throughout the room.

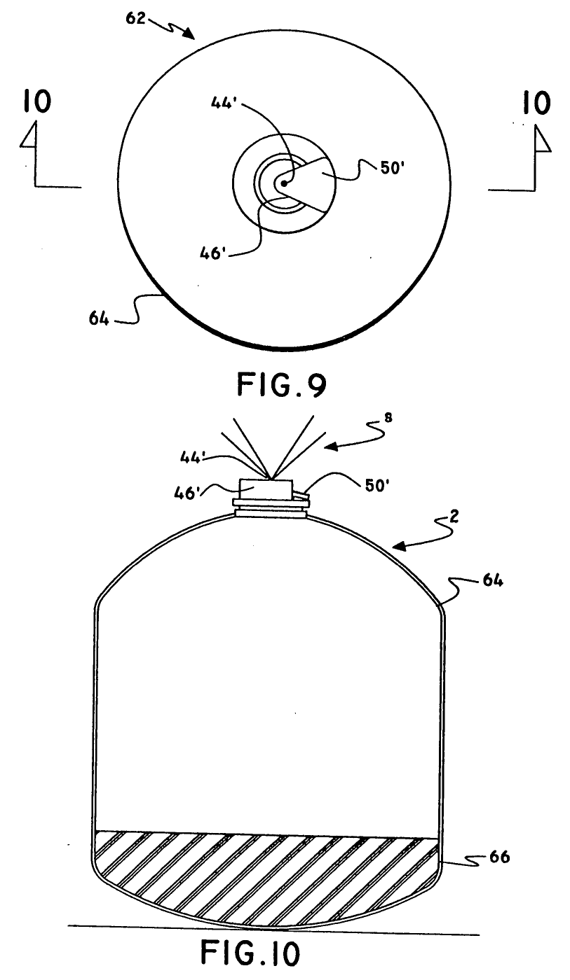

Yet another form of canister is shown in FIGS. 9 and 10, where a canister 62 is much shorter and fatter than canisters 40 and 40′, but utilizes the same valve 44′. Canister 62 has a body 64 with an integral rounded weighted base 66, made of any heavy substance, such as lead, water, sand or shot. This embodiment can likewise incorporate a closable filling aperture as in the FIGS. 7 and 8 embodiment. This embodiment is also designed to be actuated and then tossed into a room from a remote location.

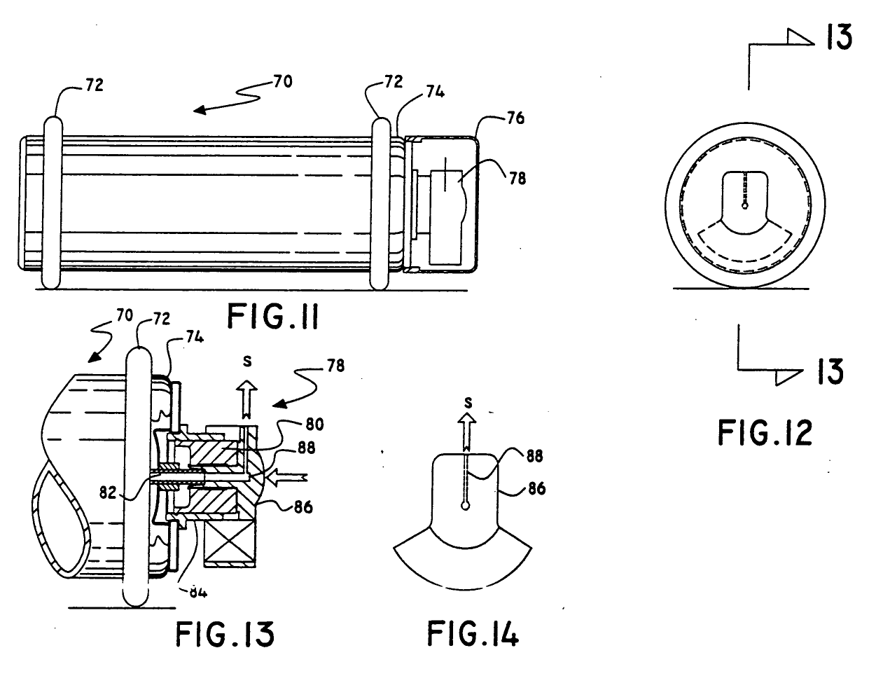

Still another embodiment is shown in FIGS. 11-14. Here a canister 70 incorporates a pair of spaced, raised circular ridges or “wheels” 72 mounted adjacent each end. Ridges 72 enable canister 70 to be rolled along the floor from a remote position to the center of a room. It can also be manually or mechanically tossed, and will eventually assume the illustrated horizontal position. The right end 74 of canister 70 mounts a removable cap 76 that covers a simple, unique dispensing valve 78, comprising a canister end fitting 80 having an exit passage 82 that is normally closed by a membrane 84. A bottom-weighted dispenser 86 has an angled dispensing passage 88 that connects with exit passage 82 when dispenser 86 is forced onto fitting 80 to pierce membrane 84. Dispenser 86 snap fits onto fitting 80 and both have a slippery interface, such as silicone coating, to enable bottom-weighted dispenser 86 to pivot, or swing like a pendulum to seek the upright-discharge position of FIG. 13. Thus cap 76 is removed, dispenser 86 is snapped onto fitting 80 and canister 70 is rocked or tossed into a room from a remote location, with the assurance that the bottom-weighted dispenser will dispense the spray S upwardly.

Thus, this invention provides a method and several embodiments of apparatus sanitizing exposed surfaces, that are subject to human contact, in a room in a short period of time by placing an aerosol canister containing sanitizing liquid and propellant in a closed room and actuating the dispensing valve to dispense the sanitizing liquid in an aerosol mist of droplets which expand to fill the room in mere minutes.

While only a preferred embodiment has been described and shown, obvious modifications are contemplated within the scope of this invention. The following claims generally recite the subject matter which Applicant claims as his exclusive rights under the Patent Laws.

1. Apparatus for sanitizing exposed surfaces in a closed room, comprising a canister which contains a quantity of sanitizing and disinfecting liquid composition and a propellant, and includes a spray nozzle having means for opening and holding open the nozzle to emit an aerosol spray of droplets upwardly to spread throughout the room and coat exposed surfaces with a thin, quick-drying film of the composition.

2. The apparatus of claim 1, wherein the liquid composition consists essentially of a n-decyl dimethyl ammonium chloride germicidal agent in an alcohol diluent.

3. The apparatus of claim 1, wherein the canister includes gravity-responsive means for assuring that the nozzle emits the spray upwardly when the canister is tossed into the room from a remote location.

4. The apparatus of claim 3, wherein canister is generally cylindrical, with the nozzle at on end, and the gravity-responsive means includes a round bottomed weight mounted at the other end of the canister.

5. The apparatus of claim 3, wherein the round-bottomed weight includes a cavity and means for filling the cavity with a weighting substance.

6. The apparatus of claim 3, wherein the canister is generally cylindrical, with a circumferential raised rib adjacent each end, which enables the canister to be rolled, and having the spray nozzle mounted at one end on a pivotal fitting that has a weight located radially opposite the nozzle, thus assuring that gravity acting on the weight positions the nozzle for emitting the spray upward.

7. A method of sanitizing a room having exposed surfaces, comprising the steps of
a. providing a canister of sanitizing and disinfecting liquid composition, said canister having an actuating valve,
b. actuating the valve and placing the canister in the room,
c. closing the room,
d. dispensing an aerosol spray of the liquid composition through the valve throughout the room to coat the exposed surfaces with a thin coat of the composition until the canister is empty, and
e. opening the room.

8. The method of claim 7, wherein the liquid composition is an-decyl, dimethyl ammonium chloride in an alcohol diluent.

9. The method of claim 7, wherein the step of actuating the valve is followed by the step of tossing the canister into the room from a remote position.

10. The method of claim 9, including the step of providing the canister with a rounded weight at one end to assure that the canister remains upright when tossed into the room.

11. The method of claim 10, including the steps of providing the rounded weight with a closable cavity, and filling the cavity with a weighting substance prior to actuating the valve.
